(19) United States
(12) Patent Application Publication
(10) Pub. No.: US 2010/0294286 A1
(43) Pub. Date: Nov. 25, 2010

(54) FLEXIBLE AND LEAKPROOF AND LEAK-RESISTANT MEDICAL BARRIERS AND SYSTEMS AND METHODS OF USE THEREOF

(75) Inventors: Alexander Harrison Bellamy, Bothell, WA (US); Richard Radford, Auburn, WA (US)

Correspondence Address: KNOBBE MARTENS OLSON & BEAR LLP, 2040 MAIN STREET, FOURTEENTH FLOOR, IRVINE, CA, 92614, US

(73) Assignee: CENORIN ,LLC, Kent, WA (US)

(21) Appl. No.: 12/812,592

(22) PCT Filed: Jan. 19, 2009
(86) PCT No.: PCT/US2009/031413

§ 371 Date: Jul. 12, 2010

Related U.S. Application Data
(60) Provisional application No. 61/022,285, filed on Jan. 18, 2008.

(51) Int. Cl. A61M 5/00

(52) U.S. Cl. 128/887

(57) ABSTRACT

A leak-resistant medical barrier is disclosed as having a sheet with a first side and a second side. The first side has an adhesive portion adapted to be disposed on a patient's skin and a release liner disposed on the adhesive portion. The second side also has an adhesive portion adapted to be disposed either on a patient's skin, on itself, or on a patient's skin and on itself and a release liner disposed on its adhesive portion. The second side adhesive portion is adapted such that when it is disposed on itself, it joins portions of surfaces of the medical barrier to form a leak-resistant seal between the surfaces, thereby providing for a medical barrier of adjustable volume and patient-adhesive contact area.

### RELATED APPLICATIONS

This application is related to and claims the benefit under 35 U.S.C. §119(e) to U.S. Provisional Patent Application Ser. No. 61/022,285 (filed Jan. 18, 2008), titled “Flexible and Leakproof and/or Leak-Resistant Medical Barrier Systems and Methods of Use,” which is hereby expressly incorporated by reference in its entirety.

### FIELD OF THE INVENTION

The present disclosure relates generally to medical barriers. Certain embodiments relate to methods of applying a medical barrier to a patient.

### DESCRIPTION OF THE RELATED ART

Hemodialysis, chemotherapy and other treatments often require patients to tolerate access ports for vascular catheters, which extend through the skin from the outside and into the body. During activities such as bathing or swimming, the access port must be protected from water in order to reduce the risks of infection and contamination. Patients with abrasions or breaks in the outer skin layer also need to protect affected skin.

Contamination can occur through air or water. Some patients, such as renal dialysis patients, are instructed not to take showers or baths. This requires patients to spend considerably more time taking care of their basic hygiene than usual. The requirement to keep access sites free from contaminants is also inconvenient at other times.

Various problems arise when devices are used to protect portions of the skin from contamination and moisture, and various available devices seek to address these problems. Single-use devices, for example, must be easy to use and inexpensive to manufacture, but must also not be prone to leaking when the patient moves as required during normal daily activities such as washing while showering.

Devices should ideally be useful in many circumstances, such as for protecting abrasions and access ports. Devices may also be useful for patients of various shapes and sizes. All devices should seek to maximize effectiveness while introducing minimal constraints and providing maximum comfort to patients during application, removal and during movements of and around the affected area. No available device provides a satisfactory solution to these problems.

### SUMMARY OF THE INVENTION

The present invention relates to a medical device and, more particularly, to leak-proof and/or leak-resistant medical protective barriers.

In accordance with one aspect of the present invention, a water-resistant medical barrier is provided having a flexible sheet with two sides, an outer side and an inner side. Each side has at least one adhesive portion and, preferably, at least one adhesive perimeter strip.

In some embodiments, the medical barrier is comprised of a rectangular sheet having two sides, an inner side and an outer side. Across one edge of one side of the sheet, the outer side, for example, is a strip of adhesive. Across at least a portion of at least one edge of is a layer of adhesive. In some embodiments, a layer of adhesive is also disposed across at least a portion of at least one of the other three edges of the other side, the inner side, for example, and, more preferably, across all three remaining edges of the other side. In some embodiments, adhesive may also be disposed across the first edge of the inner side, but need not be. Some embodiments have a single adhesive strip with a protective release liner on the superior edge of the outer side of the medical barrier and three adhesive strips with protective release liners on the inferior and two lateral edges of the inner side of the medical barrier.

Some methods of attaching the medical barrier involve a user creating a sealed and protected volume between the skin and the barrier by removing a covering release liner and then coupling the adhesive strip on the superior edge of the outer side of the sheet to the patient and by subsequently folding at least a portion of the inner side of the sheet over itself and removing a release liner covering inferior adhesive strips and attaching the inferior adhesive strip to the patient. In some embodiments, the adhesive on the two lateral edges of the inner side adheres to itself, to the patient, or to a combination of both to form a particular sealed and protected volume. In some embodiments, the protected volume and the surface area of the patient's skin contacting adhesive are adjustable. In some embodiments, adjustments are made by the user's placement of the superior and inferior adhesive strips. The lateral adhesive strips will automatically adjust to a proper contact area based on the placement of the superior and inferior strips and the distance that the patient wants to position the barrier above the skin. The lateral strips will also form lateral seals between the membrane and the patient.

In some embodiments, the waterproof or water-resistant sheet is a film, membrane, or substrate. The film, sheet, or membrane or substrate may be any of myriad of compositions suitable for short-term or long-term contact with the skin. In some embodiments, the flexible and waterproof or water-resistant sheet of material may be comprised of a thin and flexible elastic film. The flexible waterproof sheet of material may alternatively or additionally be comprised of a hydrophilic or hydrophobic material, including, but not limited to, polymers and plastics and water-vapor-breathable films, layers, and materials. The sheet or film may be clear or opaque to any degree without departing from the spirit of the present disclosure. The sheet or film may further be shaped in various ways to carry out the purposes of treatment, including, without limitation, rectangular, square, ovular, circular, or any other suitable regular or irregular shape.

Portions of the flexible and waterproof or water-resistant sheet comprise an adhesive layer or layers disposed upon the flexible waterproof or water-resistant sheet. Such layers may be comprised of, for example, without limitation, pressure-sensitive and/or thermo-sensitive, and/or radiation-sensitive adhesive materials.

In addition to the various compositions possible within the scope of the present disclosure, the adhesive may be disposed using a variety of methods in a variety of fashions and in a variety of configurations. Some embodiments may optimize the skin contact and adhesive properties of the medical protective barrier, such as adhesive strength and configuration or arrangement of the adhesive. In this manner, the protected area is best shielded from water, soap and the like when cleansing. In other embodiments, the adhesive circumscribes the protected area on the patient with the sheet covering the protected area (e.g., an introducer, or access, port). Although the medical protective barrier periphery is believed to be the optimal location for the adhesive, the adhesive may be placed on other portions of the medical protective barrier. Such alternative configurations include, but are not limited to, adhesive applied to portions, but not the entirety, of the periphery, areas extending across the medical protective barrier other than periphery, and areas within the medical protective barrier other than its periphery. Other example configurations include straight line, circular, or zigzag patterns along the periphery of the medical protective barrier. In some embodiments, adhesive is disposed on opposing sides of the film in such a manner as to be suitable for creating a protected volume or a pocket when applied to the patient's skin.

By way of example, some embodiments of the present disclosure comprise a flexible water-proof sheet of material with an adhesive layer disposed on and coupled to the sheet using any of various application techniques well known in the art. In some embodiments the adhesive layer is disposed around a perimeter of the sheet. In some embodiments, the adhesive layer is disposed in concentric parallel lines disposed around the perimeter of the sheet or film or membrane. In some embodiments, the adhesive layer is disposed in a plurality of circular patterns suitably aligned on the surface of the film or membrane. In still other embodiments, the adhesive layer is disposed randomly. In some embodiments, a plurality of adhesive layers is applied in a single or variety of patterns. Some embodiments of the present disclosure comprise a flexible water proof sheet of material with an adhesive layer on both sides and around or along a perimeter of the sheet. In some embodiments, an adhesive is coupled to opposing sides of the film. In some embodiments, a plurality of adhesive layers is applied in a plurality of patterns. One of skill in the art will recognize that the configuration of the adhesive may be varied and yet still fall within the present disclosure.

In some preferred embodiments, at least one layer of adhesive is disposed upon at least a portion of both sides of a membrane or film. In some embodiments, a release or liner layer is removably disposed on the adhesive layer such that the adhesive is protected between the membrane and the release liner until the time desired use.

Some embodiments of the present disclosure comprise a flexible waterproof material sheet of material with an adhesive layer along the perimeter of the sheet and having a release material disposed upon the adhesive layer and comprised of tabs that extend from the perimeter of the sheet. The release material can be comprised of a two-sided strip capable of being removably coupled on at least one portion to an adhesive layer. The release material may be comprised of any suitable material that is capable of being removably coupled to an adhesive layer on at least one portion and which does not adhere significantly or substantially to patient's skin on the other side. The release material is applied in such a fashion as to allow for easy application of medical protective harrier. By covering the adhesive when the device is not in use, the release liner ensures that adhesive retains its maximum and optimal properties until the time of use.

Tabs extending from the edges of the release material assist the user in using the barrier. In some embodiments, these tabs serve to ensure patient comfort is not compromised by incorrect application. In some embodiments, the release liner is configured to contain tab portions extending there from in such a manner as to aid the user in properly applying the barrier. To further facilitate these purposes, tabs may be labeled instructing user on how and in what order to remove the release liners. For example, without limitation, tabs may be configured to overlap one another such that it is difficult or impossible to easily remove one tab without first removing another tab. Tabs may also be configured such that removal of one tab automatically begins removal of another tab. In some embodiments, the tabs are labeled with instructions for the user regarding how to remove the liner or release liner and how to apply the underlying adhesive directly or indirectly to a patient's epidermis. Through such labeling or other configuration, the presently disclose medical barrier guides users to remove the tabs in the proper order to obtain the most desirable application and removal properties of a medical bandage.

The present disclosure also relates to an application method for the disclosed medical barriers. In some embodiments, this application technique comprises the following steps.
a. Removing the protective adhesive release liner from the outer side of the sheet with the single adhesive strip utilizing a peel tab.
b. Placing the exposed adhesive strip on the patient just below the site to be protected for example: a PICC line, catheter access point, wound dressing or wound access site on the patient.
c. Removing a release liner from the inside of the sheet's remaining adhesive strips using tabs.
d. Folding the sheet up and over the PICC site or device (forming a protected volume for the PICC extensions, wound dressing, wound site or other device) and attaching the inferior edge of the inner adhesive surface to the patient's skin just above the PICC, device, wound dressing or wound access site, thus minimizing the required total adhesive contact area with the patient. At this step, a user can fold the un-attached sheet forming a protected volume around the PICC, device, wound dressing or wound access site to various degrees, gathering up any slack and pinching together adjacent inner adhesive surfaces before applying to medical barrier to the skin in order to create a seal.
e. Pressing the adhesive surfaces on each side of the access site to the patient's skin and continuing to do so down each side, compressing the adhesive surface to the outer and inner surfaces of the sheet and forming a seal on the newly formed protected volume.
f. Ensuring good adhesion by again pressing the adhesive surfaces to their respective areas of adhesion.
g. The process allows the user to form a ‘minimum’ contact area on patient's skin, which is desirable for reducing skin reactions to the adhesive as well as limiting discomfort when removed.

In this way, some disclosed methods of applying a medical barrier embodied within the present disclosure allow a user to form a medical protective barrier comprised of a protected and sealed volume, or substantially void volume capable of containing, for example, a medical implement, a PICC introducer, access point, and/or extension. Furthermore, one of ordinary skill in the art will recognize that the present disclosure supports an adjustable and customizable medical barrier. The disclosed means, methods, and devices are ideal for creating a plurality of sizes of protected volumes, covering or substantially void volume using a single size of medical protective barrier. However, it should be understood that some embodiments may exclude some of the above steps or portions of the above steps.

In some embodiments, a portion of a sheet or film upon which adhesive is disposed is accessed and the adhesive is exposed by removing at least a portion of a release liner. The exposed adhesive may then be secured to a patient's body, beneath the protected area, for example, with the remainder of the device extending downward from the protected area. Next, another portion or portions of release material may be removed from adhesive disposed upon the other side of the film. Then, the remainder of the device may be folded back over the previously secured adhesive to orientate the exposed adhesive toward the patient's skin. One of ordinary skill in the art will recognize that this forms a pocket volume within the sheet material between the portions of the sheet upon which adhesive is disposed. Pressing the newly exposed adhesive against the patient's skin will fully enclose and complete the barrier pocket.

Several benefits may be achieved by application methods such as the embodiments described above: One benefit that can be achieved is that the area of adhesion to the user can be significantly reduced. This can reduce stress on the adhesive contact area when placed on curved surfaces of the body and promotes a durable seal. Some embodiments also provide enhanced freedom to move extremities or other body parts normally while showering without compromising the protective seal of the device. Some embodiments allow the user to form a free moving pouch containing the protected devise (such as PICC or IV lines) particularly at areas of an articulating joint (such as the anti-cubital or elbow area) where shear stresses on an adhesive placement near or across a joint can compromise the adhesive seal. A further benefit of a smaller surface area of contact between the adhesive and the patient in some embodiments of the methods and devices described is less patient discomfort when removing the medical protective barrier.

Some additional benefits that may be provided with the presently disclosed medical barriers involve (i) creating a sealed chamber for surrounding PICC/IV lines, wounds, and access sites that optimize the sealing area, barrier volume, length and width, etc, (ii) creating an effective medical protective barrier, and (iii) providing a medical protective barrier having enhanced applicability and usability features such as enhanced ease of application. Some disclosed embodiments also can allow for the use of a release liner comprised of tabs that further enhances applicability and usability. Some disclosed embodiments allow for reduced amount of adhesive required to assure an effective moisture barrier, with can also translate into reduced patient discomfort during removal of a medical barrier. The adhesion area, in some embodiments, comprises a substantially planar contact area between the adhesive and a patient's skin. A further benefit of the disclosed embodiments is the creation of an adjustable-sized protected volume capable of containing a PICC-line or other medical implement. These and other aspects, features and advantages of the present medical barrier will be further understood from a description of certain preferred embodiments illustrated in the attached drawings and described as follows.

### BRIEF DESCRIPTION OF THE DRAWINGS

FIG. 1 shows an outer view of a medical barrier having multiple tabs and release liners.

FIG. 2 shows an inner view of the medical barrier having multiple tabs and release liners.

FIG. 3 shows a medical barrier being applied to a patient's arm.

FIG. 4 shows a medical barrier being applied to a patient's arm.

FIG. 5 shows a user coupling a strip of adhesive on a medical barrier to another portion of the medical barrier.

FIG. 6 shows a user coupling strips of adhesive on one portion of the medical barrier to strips of adhesive on another portion of the medical barrier.

FIG. 7 shows a size-adjusted medical barrier.

FIG. 8 shows an adjustable medical barrier.

FIGS. 9A and 9B show a circular medical barrier.

### DETAILED DESCRIPTION OF THE PREFERRED EMBODIMENT

Description will now be made of embodiments of a medical barrier having features in accordance with the present invention. It should be understood that the disclosed embodiments present examples in connection with one or more preferred embodiments, and the scope of the present invention is not limited to the embodiments disclosed herein.

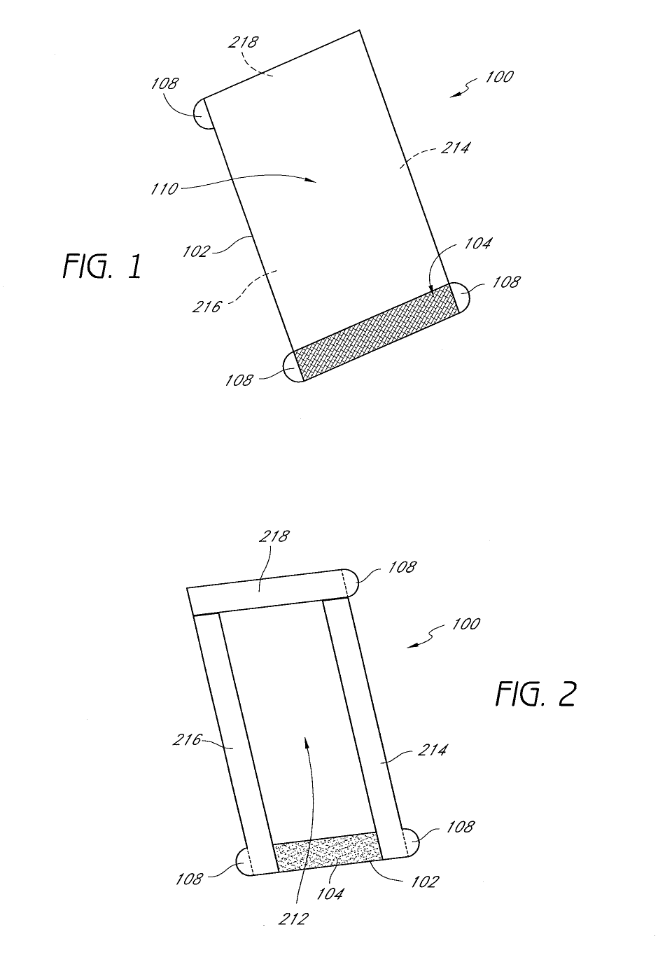

FIG. 1 shows an outer side view of an embodiment of the medical barrier 100. The barrier comprises a sheet or film 102, a plurality of adhesive layers, patches and/or strips, corresponding release liners and strategically located tabs 108. The tab portion 108 is positioned on the release liner (not shown) so as to encourage easy removal from the adhesive strip while minimizing torsion on the remainder of the sheet 102 and serving to reduce the possibility of areas of the sheet 102 sticking to one another or to themselves. The tab portion may be an integral part of any liner or a separate entity attached or attachable to any liner. As shown, a first adhesive strip 104 and corresponding release liner 106 (here removed and not shown) which may have a tab are disposed along an edge portion of the sheet's outer side 110, whereas three additional adhesive strips 424, 426, 428 (not shown) are located on the inner side 212 of the sheet 102. Each adhesive strip that is not exposed is covered by a protective release liner as, for example, the release liner 106 that is indicated (but not shown) on the sheet's outer side and that has a strategically located tab portion 108 which, in some embodiments, is integrally attached to and extends beyond sheet 102 or may be extended inward toward the center of the sheet 102.

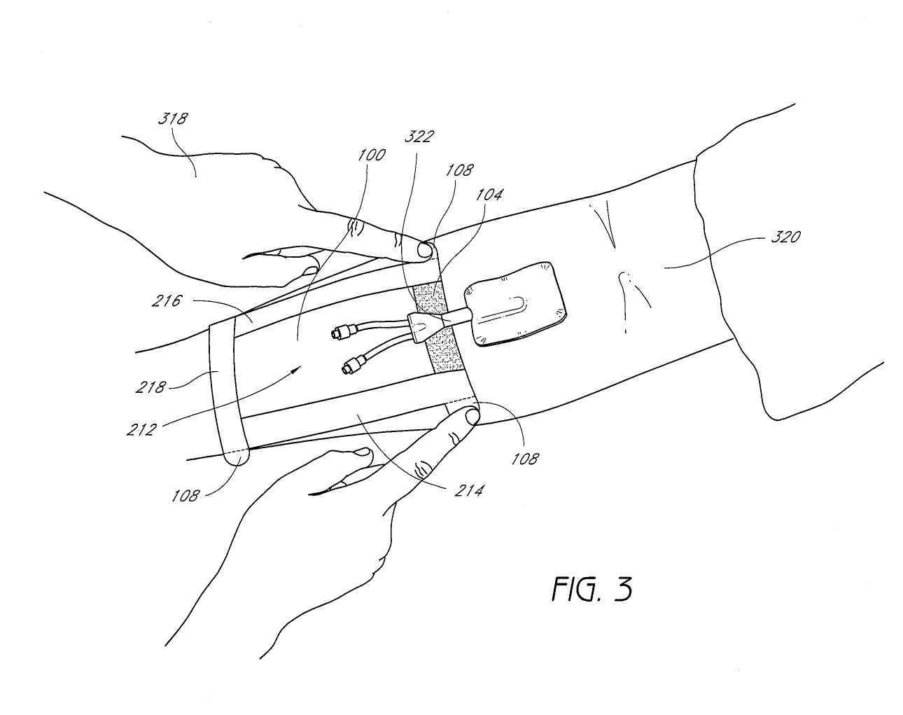

FIG. 2 shows an inner view of an embodiment of the medical barrier 100. The inner side of the barrier 100 is comprised of the inner side 212 of the sheet 102, three adhesive strips 424, 426, 428 (not shown) and three release liners, 214, 216, 218 having tabs 108. FIG. 2 further shows that a tab portion 108 may extend from any of the release liners 214, 216, 218 disposed upon the adhesive strips, 424, 426, 428 located on the inner portions of the sheet 102. These tabs are formed in similar manor to those discussed in FIG. 1 in regard to adhesive strip 104 and release liner 106. It is understood that the tabs 108 can be situated and folded in various ways to enhance user experience and to ensure that, for example, the release liner 106 is easily removed from the adhesive in the proper order to achieve maximum sealability of the medical barrier 100.

FIG. 3 shows a user 318 placing a first adhesive strip 104 on the outer portion of a medical barrier 100 (after the release liner 106 has been removed) on a patient's skin 320 below a PICC line or access port 322. Although FIG. 3 shows a PICC line or access port 322, one of ordinary skill in the art will understand that various other medical implements or treated areas may be sealed in a similar fashion. The medical barrier 100 is also comprised of at least one water-resistant sheet 102, adhesive strips 424, 426, 428 removably coupled to corresponding release liners 214, 216, 218 having tabs 108. In FIG. 3, one release liner 106 has been removed to expose the underlying adhesive 104. The access port 322 resides in the patient's body 320 and extends over the inner portion of the medical barrier 212.

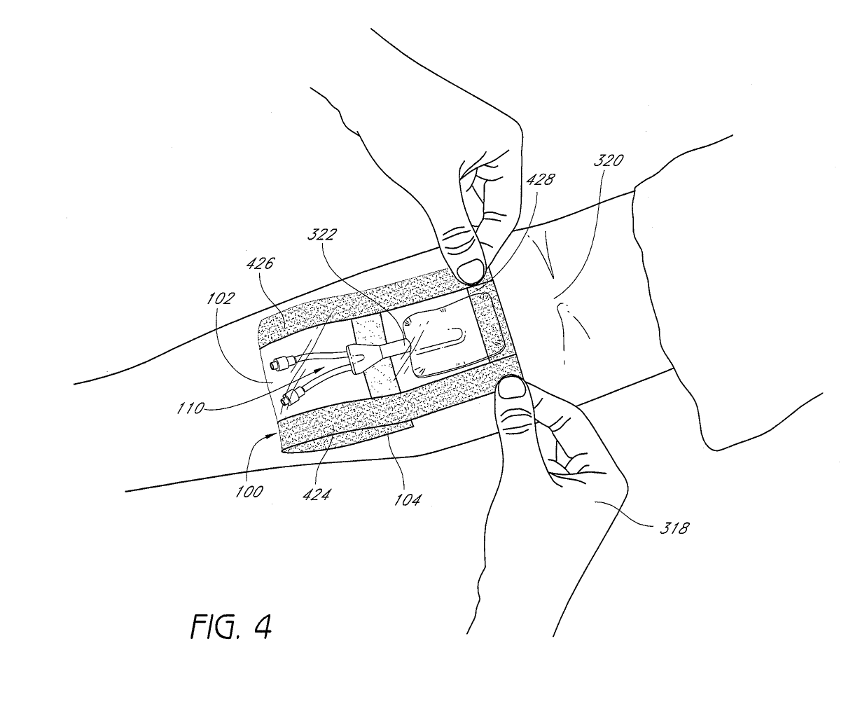

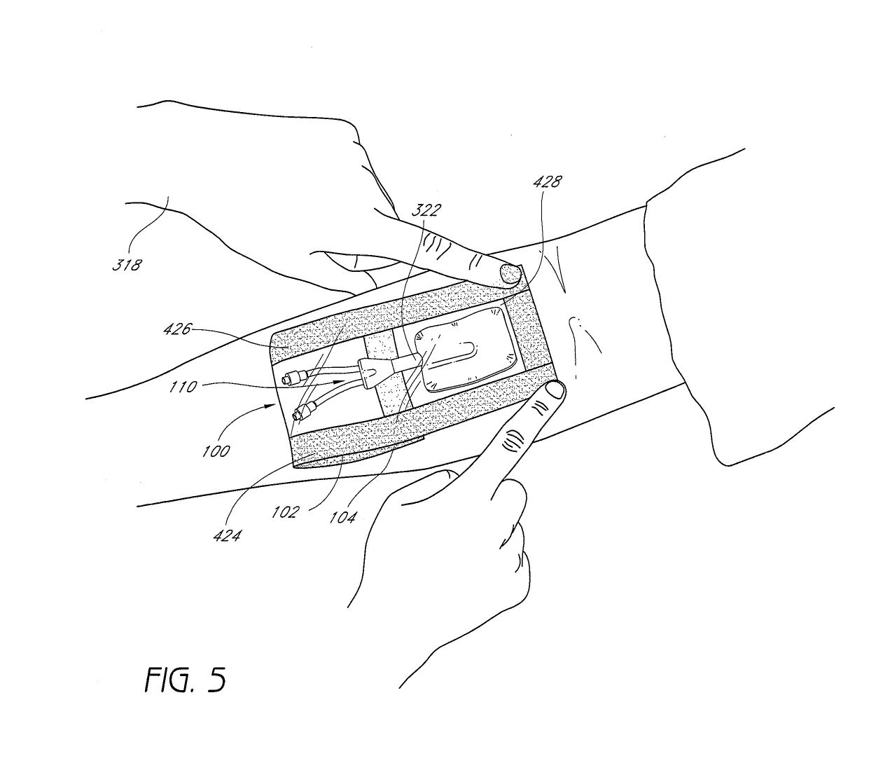

FIG. 4 shows the user 318 applying the medical barrier 100 after a first adhesive strip 104 is placed on the patient 320 below an access port 322 extending from a patient's body 320 as shown in FIG. 3. The adhesive strips on the inner side of the medical barrier 100 comprise a outer adhesive strip 428 and side adhesive strips 426, 424. The medical barrier 100 further comprises corresponding release liners 214, 216, 218 having tabs, which have been removed in this figure. The medical barrier 100 is first attached to the patient 320 as shown in FIG. 3. After the user 318 removes the remaining release liners 214, 216, 218 (not shown) by pulling on the tabs (not shown), as shown in FIG. 4, the sheet 102 is folded up and over the access port 322 to create a protected volume that is waterproof or water-resistant by sealing each of the side adhesive layers 424, 426 against itself and/or against the patient 320. The outer adhesive layer 428 is also pressed against the patient as shown in FIG. 5 completing the inner volume seal.

FIG. 5 shows the user 318 applying and sealing the sides of the medical barrier 100 after an inner superior adhesive strip 428 is placed on the patient 320. This can be done by pressing the adhesive surfaces 424, 426 on each side of the access site to the patient's skin and continuing to do so down each side, compressing the adhesive surface to the outer and inner surfaces of the sheet and forming a seal on the newly formed protected volume sides.

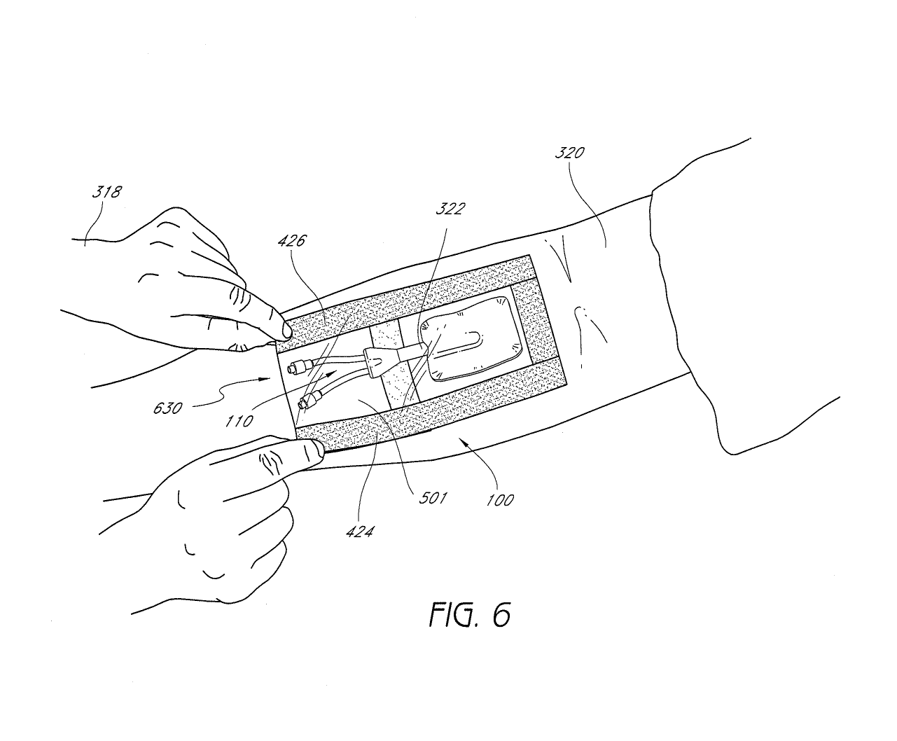

FIG. 6 shows the user 318 ensuring good adhesion by again pressing the adhesive surfaces 104, 424, 426 to their respective areas of adhesion. The user 318 compresses the adhesive surfaces 104, 424, 426, 428 of the sheet 102 against the patient 320 to form a perimeter seal around a protected volume 630 and between the patient 320 and the sheet 102. FIG. 6 depicts the free moving protected volume or pouch 501 that can be formed to hold and protect a PICC/IV device, for example, allowing for independent movement when located at a patient's joint.

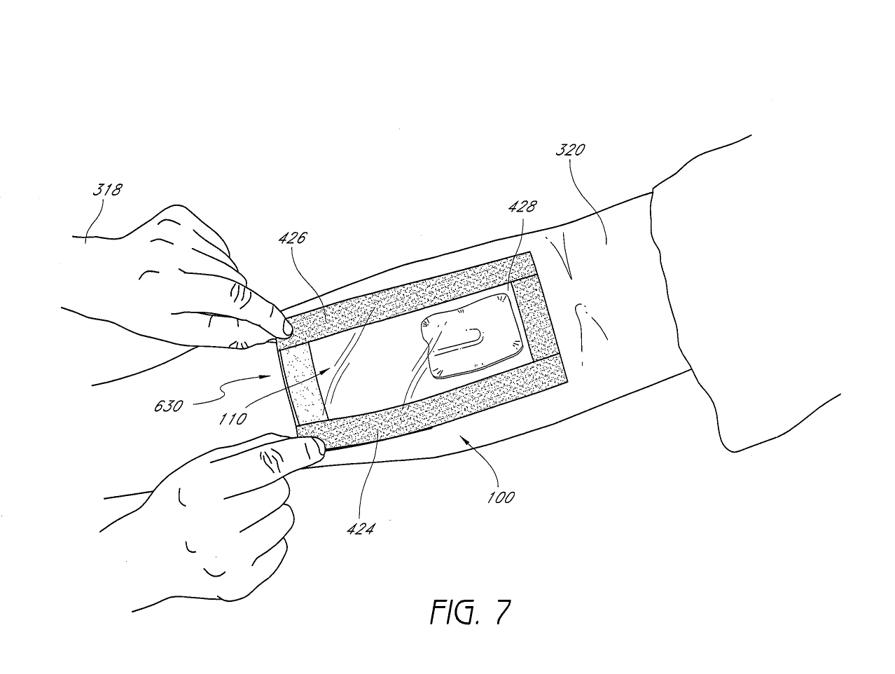

Alternatively, the adhesive layers 424 and 426 can be configured such that no slack region is left between opposite edges of the sheet 102, but such that the sheet 102 is stretched or pulled rather taut against the patient 320 as shown in FIG. 7, where a barrier 100 covering an access point on the patient 320 is covered by the medical barrier. Compared to the embodiment of FIG. 6, the proximal adhesive portion 104 in FIG. 7 adheres more proximally on the patient's appendage 320 and does not create a substantial protected volume.

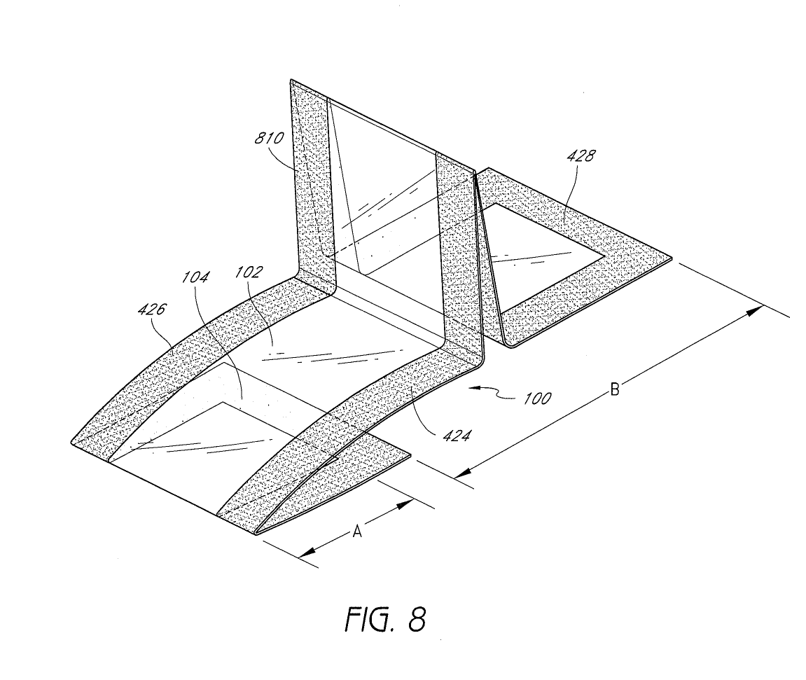

FIG. 8 shows other embodiments where the size of the protected volume can be tailored to a particular medical implement and body access port. The user can adjust the protected volume size to accommodate particular body dimensions, movement patterns, medical implements or body access ports by increasing or decreasing the amount of overlap in sheet 102 as indicated by Dimension A of FIG. 8. In some embodiments, adhesive is disposed in a strip having a width and extends along the outer side of sheet 102 along Dimension A. In some embodiments, this adhesive strip (and a corresponding release liner) extends only partially along the length of sheet 102 that includes Dimensions A and B. In some embodiments, adhesive is disposed along the entire length of sheet 102 that includes Dimensions A and B. In some embodiments, the release liner that extends along the adhesive strip that extends along Dimension A and/or Dimension B of the outer side of sheet 102 is notched or portioned. This way, the user can adjust the Dimension A without exposing more adhesive than is necessary to contact the skin.

In some embodiments, tabbed release liners, as discussed with respect to FIGS. 1 through 4 protect the adhesive along the length of sheet 102.

FIG. 8 further shows that the barrier 100 can be further modified to fit various sizes of protected volume area. For example, if the protected area is very small or substantially no volume is needed, the adhesive layers 424 and 426 can be configured such that a slack region 810 is left above the protected volume between opposite edges of the sheet 102. Where slack is left in the adhesive, the slack portions 810 can be pinched together and then pressed against the patient to create a custom-sized, less voluminous water-resistant cover over the protected area. The amount of slack region 810 then determines the volume of the protected area coverage as shown by Dimension B of FIG. 8. An advantage of this adjustability are, for example, that the adhesive-to-skin contact area surrounding the protected area can be minimized, thereby reducing pain and potential trauma or other discomfort during the removal process.

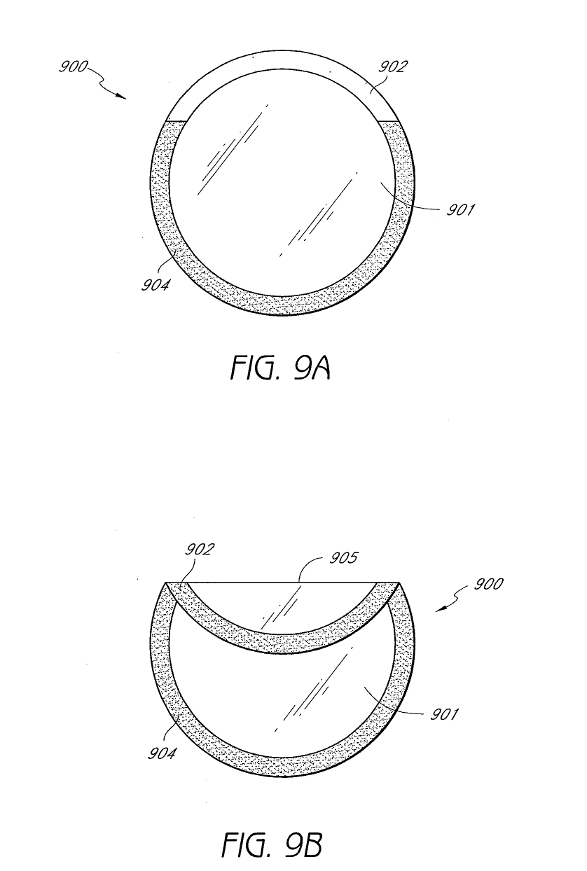

FIGS. 9A and 9B show a further embodiment in which the shape of the medical barrier is circular. The barrier 900 comprises a circular sheet 901 and inferior 902 and superior 904 adhesive layers. In some embodiments, the adhesive layers 902, 904 are covered by one or more corresponding release liners. In some embodiments, the release liners have tabs analogous to the tabs discussed with respect to the embodiments described above. FIG. 9A shows a top-side view of the sheet 901—i.e., the looking down onto the superior side of the sheet. FIG. 9B shows a bottom-side view of the sheet 901—i.e., looking up from the inferior side of the sheet—where the superior side of the sheet has been folded over the inferior side of the sheet. As shown in FIG. 9B, this embodiment can fold over itself along a fold line 905. The fold line 905 is defined by the two portions of the sheet where the inferior adhesive layer 902 and the superior adhesive layer 904 would meet if they were both on the same side of the sheet 901. The portions of the sheet 901 upon which adhesive is disposed are more rigid than the portions of the sheet 901 on which adhesive is not disposed. This leads to a preferred fold at fold line 905. Although the sheet 901 could be folded elsewhere, it will preferentially fold at the fold line. The location of the fold line can be changed by changing the locations where the adhesive is disposed on the sheet. If the inferior 902 and superior 904 adhesive layers met at the midpoint along the circumference of the sheet 901, the fold line would be along the sheet's diameter. While the concept of the fold line is discussed here with regard to the embodiment of FIGS. 9A and 9B, it is equally applicable to other embodiments, including the embodiments described above.

The embodiment shown in FIGS. 9A and 9B can be applied in a manner like the embodiments described above. Some embodiments involve first applying the inferior adhesive 902 to the patient's skin, and then folding the superior side of the sheet 901 over the inferior side of the sheet and applying the superior adhesive 904 to the patients skin. Some embodiments that have a circular sheet are adjustable based on the principles detailed above with respect to the other disclosed embodiments. Thus, this embodiment provides a further example of a medical barrier which can be used to form an adjustable pouch outside of a patient's skin.

Although certain preferred embodiments and examples have been discussed herein, it will be understood by those skilled in the art that the present invention extends beyond the specifically disclosed embodiments to other alternative embodiments and/or uses of the invention and obvious modifications and equivalents thereof. In addition, while a number of variations of the invention have been shown and described in detail, other modifications, which are within the scope of this invention, will be readily apparent to those of skill in the art based upon this disclosure. It is also contemplated that various combinations or sub-combinations of the specific features and aspects of the embodiments may be made and still fall within the scope of the invention. Accordingly, it should be understood that various features and aspects of the disclosed embodiments can be combined with or substituted for one another in order to form varying modes of the disclosed invention. Thus, it is intended that the scope of the present invention herein disclosed should not be limited by the particular disclosed embodiments described above, but should be determined only by a fair reading of the present disclosure, including the appended claims.

What is claimed is:

1. A leak-resistant medical barrier comprising:
a sheet having a first side layer and a second side layer and being foldable at multiple points along a length of the sheet;
a first adhesive portion being disposed on the first side layer of the sheet;
a second adhesive portion being disposed on the second side layer of the sheet, the first and second adhesive portions together defining an adhesive footprint when the sheet is folded, the adhesive footprint defining a closed-perimeter shape.

2. The leak-resistant medical barrier of claim 1, the first side adhesive not co-extensive in area with the sheet.

3. The leak-resistant medical barrier of claim 1, further comprising a first release liner disposed on the first adhesive portion and a second release liner disposed on the second adhesive portion.

4. The leak-resistant medical barrier of claim 1, the second adhesive portion adapted such that it can be is disposed on itself to join portions of surfaces of the medical barrier and to form a leak-resistant seal between those portions.

5. The leak-resistant medical barrier of claim 1, the first side comprising a second first side adhesive portion having adhesive thereon and extending from the first outer side adhesive portion and adapted to be disposed on the patient's skin.

6. The leak-resistant medical barrier of claim 5, the second first side adhesive portion being covered with a second first side release liner.

7. The leak-resistant medical barrier of claim 3, the release liners further comprising tab portions extending therefrom.

8. The leak-resistant medical barrier of claim 1, wherein the second adhesive portion extends about a sufficiently greater portion of the peripheral edge of the sheet than the first portion so as to form a protective volume that is adjustable.

9. The leak-resistant medical barrier of claim 8, wherein the second adhesive portion extends a portion of the peripheral edge of the sheet that is at least about 1.8 times greater than the portion of the peripheral edge of the sheet that the first portion extends about.

10. The leak-resistant medical barrier of claim 8, wherein the second adhesive portion extends a portion of the peripheral edge of the sheet that is at least about 1.75 times greater than the portion of the peripheral edge of the sheet that the first portion extends about.

11. The leak-resistant medical barrier of claim 8, wherein the second adhesive portion extends a portion of the peripheral edge of the sheet that is at least about 1.7 times greater than the portion of the peripheral edge of the sheet that the first portion extends about.

12. The leak-resistant medical barrier of claim 8, wherein the second adhesive portion extends a portion of the peripheral edge of the sheet that is at least about 1.65 times greater than the portion of the peripheral edge of the sheet that the first portion extends about.

13. The leak-resistant medical barrier of claim 8, wherein the second adhesive portion extends a portion of the peripheral edge of the sheet that is at least about 1.60 times greater than the portion of the peripheral edge of the sheet that the first portion extends about.

14. The leak-resistant medical barrier of claim 8, wherein the second adhesive portion extends a portion of the peripheral edge of the sheet that is at least about 1.55 times greater than the portion of the peripheral edge of the sheet that the first portion extends about.

15. The leak-resistant medical barrier of claim 8, wherein the second adhesive portion extends a portion of the peripheral edge of the sheet that is at least about 1.50 times greater than the portion of the peripheral edge of the sheet that the first portion extends about.

16. The leak-resistant medical barrier of claim 1, wherein the second adhesive portion is configured to form a protected volume that is adjustable in its dimensions and allows the formation of a pocket that is not adhered to the patient and can move at least partially independently from the patient to hold and protect a medical device that is attached to the patient by the first adhesive portion.

17. The leak-resistant medical barrier of claim 1, wherein the sheet is a regular geometric shape.

18. The leak-resistant medical barrier of claim 1, wherein the sheet is an irregular geometric shape.

19. The leak-resistant medical barrier of claim 1, wherein the sheet is made of water-vapor breathable material.

20. The leak-resistant medical barrier of claim 1, wherein the sheet is made of non-breathable material.

21. The leak-resistant medical barrier of claim 1, wherein at least one of the adhesive portions is composed of the same material as the at least one sheet.

22. A leak-resistant medical barrier comprising:
a sheet having a first side layer and a second side layer and being foldable at multiple points along a length of the sheet;
a first adhesive portion being disposed on the first side layer of the sheet;
a second adhesive portion being disposed on the second side layer of the sheet, at least part of the second adhesive portion being disposed directly above at least part of the first adhesive portion in a direction perpendicular to the sheet when the sheet is folded upon itself.

23. The leak-resistant medical barrier of claim 22, the first side adhesive not co-extensive in area with the sheet.

24. The leak-resistant medical barrier of claim 22, further comprising a first release liner disposed on the first adhesive portion and a second release liner disposed on the second adhesive portion.

25. The leak-resistant medical barrier of claim 24, the release liners further comprising tab portions extending therefrom.

26. The leak-resistant medical barrier of claim 22, wherein the sheet is a regular geometric shape.

27. The leak-resistant medical barrier of claim 22, wherein the sheet is an irregular geometric shape.

28. The leak-resistant medical barrier of claim 22, wherein the sheet is made of water-vapor breathable material.

29. The leak-resistant medical barrier of claim 22, wherein the sheet is made of non-breathable material.

30. A method of applying a medical barrier having a first side and a second side to a patient comprising:
removing an first side release liner from a first side adhesive portion having adhesive disposed thereon and located on the first side of the medical barrier;
applying the first side adhesive portion to the patient such that at least a portion of the first side of the medical barrier is coupled to the patient;
removing at least one second side release liner from at least one corresponding second side adhesive portion having adhesive thereon on the second side of the medical barrier to expose the second side adhesive portion having adhesive thereon; and
folding the medical barrier over at least a portion of the second side and adhering the second side adhesive portion to the patient and to a surface of the medical barrier to create a protected volume enclosed within a leak-resistant seal and defined by the second side adhesive portion of the medical barrier.

31. The method of claim 30, wherein folding the medical barrier to create the protected volume comprises pinching together a slack region between opposite edges of the at least one inner side adhesive portion to create a smaller volume enclosed within a leak-resistant seal defined by the inner side adhesive.

32. The method of claim 30, wherein removing the outer side release liner from the outer side adhesive portion further comprises folding an outer side tab portion such that the tab portion extends substantially parallel with the first adhesive portion to provide leverage in removing the outer side release liner.

33. The method of claim 30, wherein removing the inner side release liner from the inner side adhesive portion further comprises folding an inner side tab portion such that the inner side tab portion extends substantially parallel with the at least one inner side adhesive portion to provide leverage in removing the inner side release liner.

34. The method of claim 30, wherein applying the outer side adhesive portion to the patient comprises applying the outer side adhesive portion to a portion of the patient's skin that is near a medical implement extending therefrom and wherein contacting the inner side adhesive portion with a surface to create a leak-resistant seal further comprises folding the medical barrier to surround the medical implement.

35. The method of claim 30, wherein adhering the inner side adhesive strip with a surface of the medical barrier to create a leak-resistant seal comprises adhering a first portion of the inner side adhesive portion to a second portion of the inner side adhesive strip so as to create protected volume.

36. The method of claim 30, wherein the removing the at least one second side release liner step is done after applying the first side adhesive portion to the patient step.
